SEQUENCE LISTING (97 sequences)

SEQ ID NO: 1 - protein, 25 aa, Artificial Sequence
  Organism  Human immunodeficiency virus (Suc-ANYLALIEALIRAAQEQQEKNEAAL-NH2)
ANYLALIEALIRAAQEQQEKNEAAL

SEQ ID NO: 2 - protein, 26 aa, Artificial Sequence
  Organism  Human immunodeficiency virus (Ac-ANYAALIEALIRAAQEQQEKNEAALR-NH2)
ANYAALIEALIRAAQEQQEKNEAALR

SEQ ID NO: 3 - protein, 26 aa, Artificial Sequence
  Organism  Human Immunodeficiency virus (Suc-ANYAALIEALIRAAQEQQEKNEAALR-NH2)
ANYAALIEALIRAAQEQQEKNEAALR

SEQ ID NO: 4 - protein, 26 aa, Artificial Sequence
  Organism  Human immunodeficiency virus (Ac-A-C(Bzl)-YAALIEALIRAAQEQQEKNEAALR-NH2)
AXYAALIEALIRAAQEQQEKNEAALR

SEQ ID NO: 5 - protein, 26 aa, Artificial Sequence
  Organism  Human immunodeficiency virus (Ac-ANYAALIEALIRKAQEQQEKNEAALR-NH2)
ANYAALIEALIRKAQEQQEKNEAALR

SEQ ID NO: 6 - protein, 26 aa, Artificial Sequence
  Organism  Human immunodeficiency virus (Ac-ANYLALIEALIRAAQEQQEKNEAALR-NH2)
ANYLALIEALIRAAQEQQEKNEAALR

SEQ ID NO: 7 - protein, 26 aa, Artificial Sequence
  Organism  Human immunodeficiency virus (Suc-ANYLALIEALIRAAQEQQEKNEAALR-NH2)
ANYLALIEALIRAAQEQQEKNEAALR

SEQ ID NO: 8 - protein, 26 aa, Artificial Sequence
  Organism  Human immunodeficiency virus (Ac-ENYLALIEALIRAAQEQQEKNEAALR-NH2)
ENYLALIEALIRAAQEQQEKNEAALR

SEQ ID NO: 9 - protein, 26 aa, Artificial Sequence
  Organism  Human immunodeficiency virus (Ac-ENYLALIEALIRAAQEQQEKNEAALL-NH2)
ENYLALIEALIRAAQEQQEKNEAALL

SEQ ID NO: 10 - protein, 26 aa, Artificial Sequence
  Organism  Human immunodeficiency virus (Ac-ANYLALIEELIRAAQEQQEKNEAALR-NH2)
ANYLALIEELIRAAQEQQEKNEAALR

SEQ ID NO: 11 - protein, 26 aa, Artificial Sequence
  Organism  Human immunodeficiency virus (Suc-ANYLALIEAL-Tba-RAAQEQQEKNEAALR-NH2)
ANYLALIEALXRAAQEQQEKNEAALR

SEQ ID NO: 12 - protein, 26 aa, Artificial Sequence
  Organism  Human immunodeficiency virus (Suc-aNYLALIEALIRAAQEQQEKNEAALr-NH2)
ANYLALIEALIRAAQEQQEKNEAALR

SEQ ID NO: 13 - protein, 26 aa, Artificial Sequence
  Organism  Human immunodeficiency virus Suc-ANYLALIEALIRAAQEQQEKNEAALK(palmitoyl)-NH2
ANYLALIEALIRAAQEQQEKNEAALK

SEQ ID NO: 14 - protein, 26 aa, Artificial Sequence
  Organism  Human immunodeficiency virus (Pentanoyl-ANYAALIEALIRAAQEQQEKNEAALR-NH2)
ANYAALIEALIRAAQEQQEKNEAALR

SEQ ID NO: 15 - protein, 26 aa, Artificial Sequence
  Organism  Human immunodeficiency virus (Pentanoyl-ANYLALIEALIRAAQEQQEKNEAALR-NH2)
ANYLALIEALIRAAQEQQEKNEAALR

SEQ ID NO: 16 - protein, 26 aa, Artificial Sequence
  Organism  Human immunodeficiency virus (Pentanoyl-ANYAALIEALIRKAQEQQEKNEAALR-NH2)
ANYAALIEALIRKAQEQQEKNEAALR

SEQ ID NO: 17 - protein, 26 aa, Artificial Sequence
  Organism  Human immunodeficiency virus (Isovaleryl-ANYAALIEALIRAAQEQQEKNEAALR-NH2)
ANYAALIEALIRAAQEQQEKNEAALR

SEQ ID NO: 18 - protein, 26 aa, Artificial Sequence
  Organism  Human immunodeficiency virus (Isovaleryl-E-C(Bzl)-YLALIEELIRKAQEQQEKNEAALR-NH2)
EXYLALIEELIRKAQEQQEKNEAALR

SEQ ID NO: 19 - protein, 26 aa, Artificial Sequence
  Organism  Human immunodeficiency virus Suc-ENYLALIEELIRKAQEQQEKNEAALR-NH2
ENYLALIEELIRKAQEQQEKNEAALR

SEQ ID NO: 20 - protein, 26 aa, Artificial Sequence
  Organism  Human immunodeficiency virus Suc-ENYLRLIEELIRKAQEQQEKNEAALR-NH2
ENYLRLIEELIRKAQEQQEKNEAALR

SEQ ID NO: 21 - protein, 25 aa, Artificial Sequence
  Organism  Human immunodeficiency virus (Ac-CYAACIEALIRAAQEQQEKNEAALR-NH2 ({Cys1-S-B1-S-Cys5}))
CYAACIEALIRAAQEQQEKNEAALR

SEQ ID NO: 22 - protein, 25 aa, Artificial Sequence
  Organism  Human immunodeficiency virus (Ac-CYAACIEALIRAAQEQQEKNEAALR-NH2 ({Cys1-S-B21-S-Cys5}))
CYAACIEALIRAAQEQQEKNEAALR

SEQ ID NO: 23 - protein, 25 aa, Artificial Sequence
  Organism  Human immunodeficiency virus (Suc-CYLACIEELIRKAQEQQEKNEAAL-Tba-NH2 ({Cys1-S-B2-S-Cys5}))
CYLACIEELIRKAQEQQEKNEAALX

SEQ ID NO: 24 - protein, 25 aa, Artificial Sequence
  Organism  Human immunodeficiency virus (Suc-CYLACIEELIRKAQEQQEKNEAAL-Tba-NH2 ({Cys1-S-B1-S-Cys5}))
CYLACIEELIRKAQEQQEKNEAALX

SEQ ID NO: 25 - protein, 25 aa, Artificial Sequence
  Organism  Human immunodeficiency virus (Suc-CYLACIEELIRKAQEQQEKNEAAL-Tba-NH2 ({Cys1-S-B21-S-Cys5}))
CYLACIEELIRKAQEQQEKNEAALX

SEQ ID NO: 26 - protein, 25 aa, Artificial Sequence
  Organism  Human immunodeficiency virus (Suc-CYLACIEALIRAAQEQQEKNEAALR-NH2  ({Cys1-S-B1-S-Cys5}))
CYLACIEALIRAAQEQQEKNEAALR

SEQ ID NO: 27 - protein, 25 aa, Artificial Sequence
  Organism  Human immunodeficiency virus (Suc-CYLACIEELIRKAQEQQEKNEAAL-Tba-NH2 ({Cys1-S-B3-S-Cys5}))
CYLACIEELIRKAQEQQEKNEAALX

SEQ ID NO: 28 - protein, 25 aa, Artificial Sequence
  Organism  Human immunodeficiency virus (Suc-Hcy-YLA-Hcy-IEALIRAAREQQEKNEALLR-NH2 ({Hcy1-S-B1-S-Hcy5})
XYLAXIEALIRAAREQQEKNEALLR

SEQ ID NO: 29 - protein, 26 aa, Artificial Sequence
  Organism  Human immunodeficiency virus (Suc-ECYLRCIEELIRKAQEQQEKNEAALR-NH2 ({Cys2-S-B1-S-Cys6}))
ECYLRCIEELIRKAQEQQEKNEAALR

SEQ ID NO: 30 - protein, 26 aa, Artificial Sequence
  Organism  Human immunodeficiency virus (Suc-ACYLACIDELIKKAQEQQEKNEAALR-NH2 ({Cys2-S-B1-S-Cys6}))
ACYLACIDELIKKAQEQQEKNEAALR

SEQ ID NO: 31 - protein, 26 aa, Artificial Sequence
  Organism  Human immunodeficiency virus (Suc-ACYLACIEELIRKAQEQQEKNEAALR-NH2 ({Cys2-S-B1-S-Cys6}))
ACYLACIEELIRKAQEQQEKNEAALR

SEQ ID NO: 32 - protein, 30 aa, Artificial Sequence
  Organism  Human immunodeficiency virus Suc-ENYLRLIEELIRKAQEQQEKNEAALRGSGC(cholesteryl-oxycarbonylmethyl) -NH2
ENYLRLIEELIRKAQEQQEKNEAALRGSGC

SEQ ID NO: 33 - protein, 26 aa, Artificial Sequence
  Organism  Human immunodeficiency virus (Suc-ECYLACIEALIRAAQEQQEKNEAALR-NH2 ({Cys2-S-B1-S-Cys6}))
ECYLACIEALIRAAQEQQEKNEAALR

SEQ ID NO: 34 - protein, 26 aa, Artificial Sequence
  Organism  Human immunodeficiency virus (Suc-ACYLACIEELIRKAQEQQEKNEAALr-NH2 ({Cys2-S-B1-S-Cys6}))
ACYLACIEELIRKAQEQQEKNEAALR

SEQ ID NO: 35 - protein, 30 aa, Artificial Sequence
  Organism  Human immunodeficiency virus Suc-ANYLALIEALIRAAQEQQEKNEAALRGSGC(cholesteryl-oxycarbonylmethyl) -NH2
ANYLALIEALIRAAQEQQEKNEAALRGSGC

SEQ ID NO: 36 - protein, 26 aa, Artificial Sequence
  Organism  Human immunodeficiency virus (Suc-ACYLACIDELIAKAQEQQEKNEAALR-NH2 ({Cys2-S-B1-S-Cys6}))
ACYLACIDELIAKAQEQQEKNEAALR

SEQ ID NO: 37 - protein, 26 aa, Artificial Sequence
  Organism  Human immunodeficiency virus (Ac-ECYLACIEELIRKAQEQQEKNEAALR-NH2 ({Cys2-S-B1-S-Cys6}))
ECYLACIEELIRKAQEQQEKNEAALR

SEQ ID NO: 38 - protein, 26 aa, Artificial Sequence
  Organism  Human immunodeficiency virus (Pentanoyl-ECYLACIEALIRAAQEQQEKNEAALR-NH2 ({Cys2-S-B1-S-Cys6}))
ECYLACIEALIRAAQEQQEKNEAALR

SEQ ID NO: 39 - protein, 26 aa, Artificial Sequence
  Organism  Human immunodeficiency virus (Pentanoyl-ECYLACIEALIRAAQEQQEKNEAALR-NH2 ({Cys2-S-B21-S-Cys6}))
ECYLACIEALIRAAQEQQEKNEAALR

SEQ ID NO: 40 - protein, 26 aa, Artificial Sequence
  Organism  Human immunodeficiency virus (Butanoyl-ECYLACIEELIRKAQEQQEKNEAALR-NH2 ({Cys2-S-B1-S-Cys6}))
ECYLACIEELIRKAQEQQEKNEAALR

SEQ ID NO: 41 - protein, 26 aa, Artificial Sequence
  Organism  Human immunodeficiency virus (Pentanoyl-ECYLACIEELIRKAQEQQEKNEAALR-NH2 ({Cys2-S-B1-S-Cys6}))
ECYLACIEELIRKAQEQQEKNEAALR

SEQ ID NO: 42 - protein, 26 aa, Artificial Sequence
  Organism  Human immunodeficiency virus (Hexanoyl-ECYLACIEELIRKAQEQQEKNEAALR-NH2 ({Cys2-S-B1-S-Cys6}))
ECYLACIEELIRKAQEQQEKNEAALR

SEQ ID NO: 43 - protein, 26 aa, Artificial Sequence
  Organism  Human immunodeficiency virus (Suc-ACYLACIEALIRAAQEQQEKNEAALR-NH2 ({Cys2-S-B1-S-Cys6}))
ACYLACIEALIRAAQEQQEKNEAALR

SEQ ID NO: 44 - protein, 26 aa, Artificial Sequence
  Organism  Human immunodeficiency virus (Suc-ACY-Nva-ACIEALIRAAQEQQEKNEAALR-NH2 ({Cys2-S-B1-S-Cys6}))
ACYXACIEALIRAAQEQQEKNEAALR

SEQ ID NO: 45 - protein, 26 aa, Artificial Sequence
  Organism  Human immunodeficiency virus (Ac-ECYLACIEALIRAAQEQQEKNEAALR-NH2 ({Cys2-S-B1-S-Cys6}))
ECYLACIEALIRAAQEQQEKNEAALR

SEQ ID NO: 46 - protein, 26 aa, Artificial Sequence
  Organism  Human immunodeficiency virus (Suc-ACYLACIEALIRA-Nva-QEQQEKNEAALR-NH2 ({Cys2-S-B1-S-Cys6}))
ACYLACIEALIRAXQEQQEKNEAALR

SEQ ID NO: 47 - protein, 26 aa, Artificial Sequence
  Organism  Human immunodeficiency virus (Suc-ACYLACIEALIRA-Abu-QEQQEKNEAALR-NH2 ({Cys2-S-B1-S-Cys6}))
ACYLACIEALIRAXQEQQEKNEAALR

SEQ ID NO: 48 - protein, 26 aa, Artificial Sequence
  Organism  Human immunodeficiency virus (Ac-ACYLACIEALIRKAQEQQEKNEAALR-NH2 ({Cys2-S-B1-S-Cys6}))
ACYLACIEALIRKAQEQQEKNEAALR

SEQ ID NO: 49 - protein, 26 aa, Artificial Sequence
  Organism  Human immunodeficiency virus (Suc-ACY-Abu-ACIEALIRAAQEQQEKNEAALR-NH2 ({Cys2-S-B1-S-Cys6}))
ACYXACIEALIRAAQEQQEKNEAALR

SEQ ID NO: 50 - protein, 26 aa, Artificial Sequence
  Organism  Human immunodeficiency virus (Ac-ECYLRCIEALIRAAQEQQEKNEAALR-NH2  ({Cys2-S-B1-S-Cys6}))
ECYLRCIEALIRAAQEQQEKNEAALR

SEQ ID NO: 51 - protein, 26 aa, Artificial Sequence
  Organism  Human immunodeficiency virus (Ac-ACYLACIEALIRRAQEQQEKNEAALR-NH2 ({Cys2-S-B1-S-Cys6}))
ACYLACIEALIRRAQEQQEKNEAALR

SEQ ID NO: 52 - protein, 26 aa, Artificial Sequence
  Organism  Human immunodeficiency virus Pentanoyl-ENYLALIEELIRKAQEQQEKNEAALR-NH2
ENYLALIEELIRKAQEQQEKNEAALR

SEQ ID NO: 53 - protein, 26 aa, Artificial Sequence
  Organism  Human immunodeficiency virus (Ac-ECYAACIEELIRKAQEQQEKNEAALR-NH2 ({Cys2-S-B1-S-Cys6}))
ECYAACIEELIRKAQEQQEKNEAALR

SEQ ID NO: 54 - protein, 26 aa, Artificial Sequence
  Organism  Human immunodeficiency virus (Ac-ACYLACIEALIRAAQEQQEKNEAALL-NH2 ({Cys2-S-B1-S-Cys6}))
ACYLACIEALIRAAQEQQEKNEAALL

SEQ ID NO: 55 - protein, 30 aa, Artificial Sequence
  Organism  Human immunodeficiency virus Suc-ENYLRLIEELIRKAQEQQEKNEAALRGSGK(palmitoyl)-NH2
ENYLRLIEELIRKAQEQQEKNEAALRGSGK

SEQ ID NO: 56 - protein, 26 aa, Artificial Sequence
  Organism  Human immunodeficiency virus (Ac-ACYLACIEALIRAAQEQQEKNEAALR-NH2 ({Cys2-S-B1-S-Cys6}))
ACYLACIEALIRAAQEQQEKNEAALR

SEQ ID NO: 57 - protein, 26 aa, Artificial Sequence
  Organism  Human immunodeficiency virus (Ac-ACYIACIEALIRAAQEQQEKNEAALR-NH2 ({Cys2-S-B1-S-Cys6}))
ACYIACIEALIRAAQEQQEKNEAALR

SEQ ID NO: 58 - protein, 26 aa, Artificial Sequence
  Organism  Human immunodeficiency virus (Ac-ACYLACIEALIRAAQEQQEKNEAALR-NH2 ({Cys2-S-B21-S-Cys6}))
ACYLACIEALIRAAQEQQEKNEAALR

SEQ ID NO: 59 - protein, 26 aa, Artificial Sequence
  Organism  Human immunodeficiency virus (Ac-ACY-Cha-ACIEALIRAAQEQQEKNEAALR-NH2 ({Cys2-S-B1-S-Cys6}))
ACYXACIEALIRAAQEQQEKNEAALR

SEQ ID NO: 60 - protein, 26 aa, Artificial Sequence
  Organism  Human immunodeficiency virus (Ac-ACY-Tba-ACIEALIRAAQEQQEKNEAALR-NH2 ({Cys2-S-B1-S-Cys6}))
ACYXACIEALIRAAQEQQEKNEAALR

SEQ ID NO: 61 - protein, 26 aa, Artificial Sequence
  Organism  Human immunodeficiency virus (Ac-ACYFACIEALIRAAQEQQEKNEAALR-NH2 ({Cys2-S-B1-S-Cys6}))
ACYFACIEALIRAAQEQQEKNEAALR

SEQ ID NO: 62 - protein, 26 aa, Artificial Sequence
  Organism  Human immunodeficiency virus (Ac-ACYVACIEALIRAAQEQQEKNEAALR-NH2 ({Cys2-S-B1-S-Cys6}))
ACYVACIEALIRAAQEQQEKNEAALR

SEQ ID NO: 63 - protein, 26 aa, Artificial Sequence
  Organism  Human immunodeficiency virus (Ac-ACYWACIEALIRAAQEQQEKNEAALR-NH2 ({Cys2-S-B1-S-Cys6}))
ACYWACIEALIRAAQEQQEKNEAALR

SEQ ID NO: 64 - protein, 26 aa, Artificial Sequence
  Organism  Human immunodeficiency virus (Ac-ACYAACIEALIRAAQEQQEKNEAALR-NH2({Cys2-S-B3-S-Cys6}))
ACYAACIEALIRAAQEQQEKNEAALR

SEQ ID NO: 65 - protein, 26 aa, Artificial Sequence
  Organism  Human immunodeficiency virus (Ac-ACYAACIEALIRAAQEQQEKNEAALR-NH2({Cys2-S-B2-S-Cys6}))
ACYAACIEALIRAAQEQQEKNEAALR

SEQ ID NO: 66 - protein, 26 aa, Artificial Sequence
  Organism  Human immunodeficiency virus (Ac-ACYAACIEALIRAAQEQQEKNEAALR-NH2 ({Cys2-S-B21-S-Cys6}))
ACYAACIEALIRAAQEQQEKNEAALR

SEQ ID NO: 67 - protein, 26 aa, Artificial Sequence
  Organism  Human immunodeficiency virus (Suc-ACYAACIEALIRAAQEQQEKNEAALR-NH2 ({Cys2-S-B1-S-Cys6}))
ACYAACIEALIRAAQEQQEKNEAALR

SEQ ID NO: 68 - protein, 26 aa, Artificial Sequence
  Organism  Human immunodeficiency virus (Ac-ACYAACIEALIRAAQEQQEKNEAALR-NH2 ({Cys2-S-B1-S-Cys6}))
ACYAACIEALIRAAQEQQEKNEAALR

SEQ ID NO: 69 - protein, 30 aa, Artificial Sequence
  Organism  Human immunodeficiency virus Ac-ANYLALIEALIRAAQEQQEKNEAALRGSGK(cholesteryl-succinyl)-NH2
ANYLALIEALIRAAQEQQEKNEAALRGSGK

SEQ ID NO: 70 - protein, 26 aa, Artificial Sequence
  Organism  Human immunodeficiency virus (Ac-ACYAACIDALIRAAQEQQEKNEAALR-NH2 ({Cys2-S-B1-S-Cys6}))
ACYAACIDALIRAAQEQQEKNEAALR

SEQ ID NO: 71 - protein, 26 aa, Artificial Sequence
  Organism  Human immunodeficiency virus (Ac-ACYAACIEALIRKAQEQQEKNEAALR-NH2 ({Cys2-S-B1-S-Cys6}))
ACYAACIEALIRKAQEQQEKNEAALR

SEQ ID NO: 72 - protein, 26 aa, Artificial Sequence
  Organism  Human immunodeficiency virus (Ac-ACYAACIEELIRKAQEQQEKNEAALR-NH2 ({Cys2-S-B1-S-Cys6}))
ACYAACIEELIRKAQEQQEKNEAALR

SEQ ID NO: 73 - protein, 26 aa, Artificial Sequence
  Organism  Human immunodeficiency virus (Ac-ACYAACIEALIRAAQEQQEKNEALLR-NH2 ({Cys2-S-B1-S-Cys6}))
ACYAACIEALIRAAQEQQEKNEALLR

SEQ ID NO: 74 - protein, 26 aa, Artificial Sequence
  Organism  Human immunodeficiency virus (Ac-ECYAACIEALIRAAQEQQEKNEAALR-NH2 ({Cys2-S-B1-S-Cys6}))
ECYAACIEALIRAAQEQQEKNEAALR

SEQ ID NO: 75 - protein, 26 aa, Artificial Sequence
  Organism  Human immunodeficiency virus (Ac-aCYAACIEALIRAAQEQQEKNEAALr-NH2 ({Cys2-S-B1-S-Cys6}))
ACYAACIEALIRAAQEQQEKNEAALR

SEQ ID NO: 76 - protein, 26 aa, Artificial Sequence
  Organism  Human immunodeficiency virus (Ac-aCYAACIEALIRAAQEQQEKNEAALr-NH2 ({Cys2-S-B21-S-Cys6}))
ACYAACIEALIRAAQEQQEKNEAALR

SEQ ID NO: 77 - protein, 26 aa, Artificial Sequence
  Organism  Human immunodeficiency virus (Ac-A-Hcy-YAA-Hcy-IEALIRAAQEQQEKNEAALR-NH2 ({Hcy2-S-B1-S-Hcy6})
AXYAAXIEALIRAAQEQQEKNEAALR

SEQ ID NO: 78 - protein, 26 aa, Artificial Sequence
  Organism  Human immunodeficiency virus (Ac-A-Hcy-YAA-Hcy-IEALIRAAQEQQEKNEAALR-NH2 ({Hcy2-S-B21-S-Hcy6})
AXYAAXIEALIRAAQEQQEKNEAALR

SEQ ID NO: 79 - protein, 26 aa, Artificial Sequence
  Organism  Human immunodeficiency virus (Ac-A-Hcy-YAA-Hcy-IEALIRKAQEQQEKNEAALR-NH2 ({Hcy2-S-B21-S-Hcy6})
AXYAAXIEALIRKAQEQQEKNEAALR

SEQ ID NO: 80 - protein, 26 aa, Artificial Sequence
  Organism  Human immunodeficiency virus (Ac-A-Hcy-YAA-Hcy-IEALIRKAQEQQEKNEAALR-NH2 ({Hcy2-S-B21-S-Hcy6})
AXYAAXIEALIRKAQEQQEKNEAALR

SEQ ID NO: 81 - protein, 30 aa, Artificial Sequence
  Organism  Human immunodeficiency virus (Ac-ACYAACIEALIRAAQEQQEKNEAALRGSGC(cholesteryl-oxycarbonylmethyl) -NH2 ({Cys2-S-B1-S-Cys6}))
ACYAACIEALIRAAQEQQEKNEAALRGSGC

SEQ ID NO: 82 - protein, 30 aa, Artificial Sequence
  Organism  Human immunodeficiency virus (Suc-ECYLRCIEELIRKAQEQQEKNEAALRGSGC(cholesteryl-oxycarbonylmethyl )-NH2 ({Cys2-S-B1-S-Cys6}))
ECYLRCIEELIRKAQEQQEKNEAALRGSGC

SEQ ID NO: 83 - protein, 26 aa, Artificial Sequence
  Organism  Human immunodeficiency virus Ac-ACYAAcIEALIRAAQEQQEKNEAALR-NH2
ACYAACIEALIRAAQEQQEKNEAALR

SEQ ID NO: 84 - protein, 26 aa, Artificial Sequence
  Organism  Human immunodeficiency virus Ac-ACYAACIEALIRAAREQQEKNEAALR-NH2
ACYAACIEALIRAAREQQEKNEAALR

SEQ ID NO: 85 - protein, 26 aa, Artificial Sequence
  Organism  Human immunodeficiency virus Butanoyl-ECYLACIEELIRKAQEQQEKNEAALR-NH2
ECYLACIEELIRKAQEQQEKNEAALR

SEQ ID NO: 86 - protein, 26 aa, Artificial Sequence
  Organism  Human immunodeficiency virus Hexanoyl-ECYLACIEELIRKAQEQQEKNEAALR-NH2
ECYLACIEELIRKAQEQQEKNEAALR

SEQ ID NO: 87 - protein, 30 aa, Artificial Sequence; identical to SEQ ID NO: 81
  Organism  Human immunodeficiency virus Ac-ACYAACIEALIRAAQEQQEKNEAALRGSGC(cholesteryl-oxycarbonylmethyl)- NH2

SEQ ID NO: 88 - protein, 30 aa, Artificial Sequence
  Organism  Human immunodeficiency virus Suc-ECYLRCIEELIRKAQEQQEKNEAALRGSGK(palmitoyl)-NH2
ECYLRCIEELIRKAQEQQEKNEAALRGSGK

SEQ ID NO: 89 - protein, 26 aa, Artificial Sequence
  Organism  Human immunodeficiency virus Ac-ACYAACIEALIRAAQEQQEKNEAALR-NH2
ACYAACIEALIRAAQEQQEKNEAALR

SEQ ID NO: 90 - protein, 26 aa, Artificial Sequence
  Organism  Human immunodeficiency virus Suc-ECYLACIEELIRKAQEQQEKNEAALR-NH2
ECYLACIEELIRKAQEQQEKNEAALR

SEQ ID NO: 91 - protein, 26 aa, Artificial Sequence
  Organism  Human immunodeficiency virus Pentanoyl-ECYLRCIEELIRKAQEQQEKNEAALR-NH2
ECYLRCIEELIRKAQEQQEKNEAALR

SEQ ID NO: 92 - protein, 26 aa, Artificial Sequence
  Organism  Human immunodeficiency virus Pentanoyl-ECYLCLIEELIRKAQEQQEKNEAALR-NH2
ECYLCLIEELIRKAQEQQEKNEAALR

SEQ ID NO: 93 - protein, 36 aa, Artificial Sequence
  Organism  Human immunodeficiency virus Ac-YTSLIHSLIEESQNQQEKNEQELLELDKWASLWNWF-NH2
YTSLIHSLIEESQNQQEKNEQELLELDKWASLWNWF

SEQ ID NO: 94 - protein, 34 aa, Artificial Sequence
  Organism  Human immunodeficiency virus Ac-WMEWDREINNYTSLIHSLIEESQNQQEKNEQELL-NH2
WMEWDREINNYTSLIHSLIEESQNQQEKNEQELL

SEQ ID NO: 95 - protein, 26 aa, Artificial Sequence
  Organism  Human immunodeficiency virus Ac-NNYTSLIHSLIEESQNQQEKNEQELL-NH2    (PBD truncated C34)
NNYTSLIHSLIEESQNQQEKNEQELL

SEQ ID NO: 96 - protein, 36 aa, Artificial Sequence
  Organism  Human immunodeficiency virus Ac-SWETWEREIENYTRQIYRILEESQEQQDRNERDLLE-NH2
SWETWEREIENYTRQIYRILEESQEQQDRNERDLLE

SEQ ID NO: 97 - protein, 26 aa, Artificial Sequence
  Organism  Human immunodeficiency virus Ac-ENYTRQIYRILEESQEQQDRNERDLL-NH2   (PBD truncated Sifuvirtide)
ENYTRQIYRILEESQEQQDRNERDLL

(19) United States
(12) Patent Application Publication
(10) Pub. No.: US 2014/0357577 A1
(43) Pub. Date: Dec. 4, 2014

(54) HIV Membrane Fusion Inhibitors

(71) Applicants: Janssen R&D Limited, Little Island, Co Cork (IE); PEPSCAN Systems BV, Lelystad (NL)

(72) Inventors: Bruce Albert Malcolm, Springfield, NJ (US); Johannes Wilhelmus J. Thuring, Beerse (BE); Christophe Francis Robert Nestor Buyck, Hamme (BE); Wim Bert Griet Schepens, Mechelen (BE); Maria Aldegonda Jacoba Kriek, Lelystad (NL); Wilhelmus Martinus Maria Schaaper, Almere (NL); Jelle Wouter Slootstra, Lelystad (NL); Peter Timmerman, Lelystad (NL)

(21) Appl. No.: 14/367,197

(22) PCT Filed: Dec. 18, 2012
(86) PCT No.: PCT/EP2012/075956

§ 371 Date: Jun. 19, 2014

(30) Foreign Application Priority Data
Dec. 19, 2011 (EP) 11194340.3

(51) Int. Cl. C07K 14/00

(52) U.S. Cl. CPC C07K 14/001; C07K 14/00
USPC 514/21.3; 530/325; 514/21.4; 530/324

(57) ABSTRACT

The present invention concerns an inhibitor of Human Immunodeficiency Virus (HIV) fusion with, or HIV entry in, a host cell comprising at least 24, but preferably 26, contiguous amino acids; the invention also relates to a pharmaceutical composition comprising said amino acids.

The present invention concerns an inhibitor of Human Immunodeficiency Virus (HIV) fusion with, or HIV entry in, a host cell comprising at least 24, but preferably 26, contiguous amino acids; the invention also relates to a pharmaceutical composition comprising said amino acids.

Current therapy for the treatment of HIV generally targets the viral enzymes reverse transcriptase and/or protease. However, several other enzymes or structural proteins of HIV, such as the envelope glycoprotein, also play critical roles in infection.

The HIV envelope glycoprotein consists of two associated subunits, gp120 and gp41, generated by proteolytic cleavage of the precursor gp160 protein. It resides in the viral membrane as a complex of three gp120 and three gp41 subunits. It is the gp41 subunit that mediates fusion of the membranes of the virus and target cell, allowing the HIV to infect new cells. The gp120 subunit is involved in target cell recognition and receptor binding.

The process of membrane fusion mediated by gp41 involves a conformational change in the glycoprotein, which allows the N-terminal regions of the trimeric gp41 (N-helix) to penetrate the cell membrane. Following this insertion, the C-terminal regions of the three-gp41 subunits (the C-helix) fold back on the N-helix. The resulting hexameric alpha helical interaction, called the 6-helix bundle, between the N-helix and the C-helix regions of gp41, leads to close approximation of the cell and viral membranes, which eventually results in fusion of the viral and cellular membranes.

Inhibition of the formation of this stable 6-helix bundle offers an interesting approach to prevent HIV infection. The HIV envelope is composed of a lipid bilayer bearing envelope proteins composed of heavily glycosylated gp120 proteins on the exterior and gp41 transmembrane glycoproteins. The molecular sequence of gp41 includes so-called “heptad-repeat” regions (HR1 and HR2). A heptad-repeat is a structural motif that consists of a repeating pattern of seven amino acids. Entry of HIV into the host cell begins with the binding of gp120 to the cellular CD4 receptor and its subsequent binding to the chemokine co-receptors CCR5 or CXCR4. This triggers a series of conformational changes that unmasks the gp41 fusion domain, which inserts in the cell membrane. The HR2 regions then bind to the hydrophobic grooves formed by the corresponding HR1 regions, resulting in said stable 6-helix bundle. This brings viral and cellular membranes into proximity for fusion and entry. Hence, interfering with 6-helix bundle formation prevents the virus from entering the cell.

Gp41 of HIV contains two stretches of peptide, called HR1 and HR2 that form said 6-helix bundle, the formation of which is the driving force behind fusion of the viral membrane with the membrane of the host cell. The actual 6-helix bundle consists of 3 parallel stretches of HR1, the inner coiled coil, complemented on the outside, along the grooves of the inner coiled coil in an antiparallel way, by 3 stretches of HR2.

So called N36 (SGIVQQQNNLLRAIEAQQHLLQLTVWGIKQLQARIL) is part of HR1 and so called C34 (WMEWDREINNYTSLIHSLIEESQNQQEKNEQELL) is part of HR2. Most of the current peptidic fusion inhibitors are HR2 mimics, and are analogues of, or contain parts of C34. The antiviral potential of peptidic HR1 mimics is also documented and all contain the last 17 amino acids of N36, also called N17 (LLQLTVWGIKQLQARIL). The N36 derived peptides are usually fused to a peptidic tag that is also a trimeric coiled coil and has favorable solubility characteristics, partly because they contain many charged side chains. The fusion is made in such a way that the heptad repeat, typical of coiled coil zippers, is respected. Two examples of such peptidic tags are the so-called IQ sequence: (RMKQIEDKIEEIESKQKKIENEIARIKK) and the so-called IZ sequence: (IKKEIEAIKKEQEAIKKKIEAIEK). In experiments with the purpose of finding small molecules that inhibit HIV fusion, people skilled in the art tested numerous peptides, some of which were derived from published work on HR1 mimics.

One of the problems with the so-called HIV entry or HIV fusion peptides known in the art is the relative low antiviral activity of those peptides. Another problem with those peptides, especially when formulation is due into an appropriate pharmaceutical composition, is the poor solubility due to the presence of hydrophobic amino acids in said peptides. As a consequence pharmaceutical compositions comprising those peptides are hard to formulate and consequently to develop.

Furthermore it is believed in the art that the so-called HIV entry or HIV fusion peptides for the optimal antiviral activity should contain the so-called “Kim pocket” binding motif at the N-terminal side or the lipid binding motif at the C-terminal side. Both sites are considered indispensable for antiviral activity. (Eckert and Kim, PNAS, 2001, vol. 98, no 20, pp 11187-11192)

However, there remains an unmet medical need for the so-called fusion or entry HIV inhibitors based on peptides and/or on small molecules which possess both a high antiviral activity and an acceptable solubility as well for pharmaceutical formulation purposes.

In accordance with the present invention unexpectedly derivatives of a peptide, not containing the so-called Kim pocket binding motif at the N-terminal side nor the lipid binding motif at the C-terminal side, comprising at least 24 contiguous amino acids linked to a N-capping group wherein said N-capping group is selected from the group succinyl, acetyl, butanoyl, pentanoyl, hexanoyl or isovaleryl and wherein the first of said 24 amino acids is either directly linked to the N-capping group or is indirectly linked to said N-capping group via an additional amino acid selected from the group E, A or a, have shown an extremely good potency with EC₅₀ in the low nM range.

The length of the peptides of the invention are at least 24 contiguous amino acids long and linked to a N-capping group wherein said N-capping group is selected from the group succinyl, acetyl, butanoyl, pentanoyl, hexanoyl or isovaleryl and wherein the first of said 24 amino acids is either directly linked to the N-capping group or is indirectly linked to said N-capping group via an additional amino acid selected from the group E, A or a and wherein
the first amino acid is C, Hcy, C(Bzl) or N,
the second amino acid is Y,
the third amino acid is a lipophilic amino acid,
the fourth amino acid represents A or R,
the fifth amino acid is C, Hcy or L,
the sixth amino acid is I,

the seventh amino acid is an acidic amino acid,
the eighth amino acid represents alanine or an acidic amino acid,
the ninth amino acid is L,
the tenth amino acid is a lipophilic amino acid,
the eleventh amino acid is a basic amino acid,
the twelfth amino acid is alanine or a basic amino acid,
the thirteenth amino acid is a lipophilic amino acid,
the fourteenth amino acid is Q or R,
the fifteenth amino acid is E,
the sixteenth amino acid is Q,
the seventeenth amino acid is Q,
the eighteenth amino acid is E,
the nineteenth amino acid is K,
the twentieth amino acid is N,
the twenty-first amino acid is E,
the twenty-second amino acid is A,
the twenty-third amino acid is a lipophilic amino acid and
the twenty-fourth amino acid is L,
optionally said twenty-fourth amino acid is linked to an amino acid selected from the group R, r, L, Tba or K(palmitoyl).

In a further embodiment the invention relates to a peptide above mentioned wherein the third amino acid is selected from the group A, L, I, F, V, W, Tba, Nva, Abu or Cha,
the fourth amino acid is R or A,
the seventh amino acid is selected from E or D,
the eighth amino acid, when an acidic amino acid, represents E,
the tenth amino acid is selected from I or Tba,
the eleventh amino acid, when a basic amino acid, is R or K,
the twelfth amino acid, when a basic amino acid, is R or K,
the thirteenth amino acid is selected from A, Nva or Abu, and
the twenty-third amino acid is A or L.

In another embodiment the current invention concerns a peptide as defined above wherein the first amino acid and fifth amino acid independently from one another are either C or Hcy, and wherein said first and said fifth amino acid are connected via B1, B2, B3, B21 or B22. When attached to these B1, B2, B3, B21 or B22 moieties (the meaning of these abbreviations for the corresponding bridge structures, see below) a peptide according to the invention having a looped peptide structure (i−i+4 side-chain to side-chain) at its N-terminal part, is obtained: a so-called CLIPS peptide (Chemically Linked Peptides onto a (hetero) aromatic Scaffold)

The peptides according to the invention may be directly or indirectly bound at the C-terminal amino acid to cholesterol or palmitoyl or their derivatives thereof. Alternatively, they may be connected by a linker comprising two or more amino acids. Preferably the linker consists of two, three or four amino acids, more preferably four amino acids. The amino acids may be naturally occurring or synthetic amino acids. The linker may preferably comprise Gly-Ser-Gly-Cys (-GSGC-) or Gly-Ser-Gly-Lys (-GSGK-).

So part of the invention is also a peptide as mentioned above wherein the amino acid R, as linked to the twenty-fourth amino acid, is indirectly attached to cholesterol or a derivative thereof, or is indirectly attached to palmitoyl or a derivative thereof. Cholesterol is bound via acetyl to the side chain of a C-terminal cystein-amide or homocystein-amide, i.e. the linker for attachment to the cholesterol must have a cystein-amide or a homocystein-amide at its C-terminal side (see FIG. 2 below).

Said amino acid R and said cholesterol or derivative thereof are preferably linked by a linker having two or more amino acids, preferably two, three or four is amino acids, more preferably four amino acids such as -Gly-Ser-Gly-Cys- (-GSGC-).

Alternatively said amino acid R and said palmitoyl or derivative thereof are preferably linked by a linker having two or more amino acids, preferably two, three or four amino acids, more preferably four amino acids such as -Gly-Ser-Gly-Lys- (-GSGK-).

The peptides according to the invention comprise an amino acid sequence which is as such in a dimer or trimer configuration. An example is that the peptides of the invention are chemically linked to each other by for instance an -S-S- bridge.

Preferred peptides according to the invention have the amino acid sequence selected from the group: Pentanoyl-ECYLACIEALIRAAQEQQEKNEAALR-NH₂ wherein the cysteine (C) moieties in the peptide are connected via B1 and Pentanoyl-ECYLACIEELIRKAQEQQEKNEAALR-NH₂ wherein the cysteine (C) moieties in the peptide are connected via B1 respectively.

Another preferred peptide according to the invention is Suc-ECYLRCIEELIRKAQEQQEKNEAALR-NH₂ wherein the cysteine (C) moieties in the peptide are connected via B1.

Another preferred peptide according to the invention has the amino acid sequence: Isovaleryl-E-C(Bzl)-YLALIEELIRKAQEQQEKNEAALR-NH₂.

Peptides having the amino acid sequence selected from Suc-ECYLRCIEELIRKAQEQQEKNEAALRGSGC(cholesteryl-oxycarbonylmethyl)-NH₂ and Ac-ACYAACIEALIRAAQEQQEKNEAALRGSGC(cholesteryl-oxycarbonylmethyl)-NH₂ wherein the cysteine (C) moieties at position 1 and position 5 in said peptides are connected via B1 or B3 are also highly preferred, whereas B1 is the most preferred connection.

Very preferred is the peptide having the amino acid sequence Suc-ECYLRCIEELIRKAQEQQEKNEAALRGSGC(cholesteryl-oxycarbonylmethyl)-NH₂ wherein the cysteine (C) moieties at position 1 and position 5 in said peptide is connected via B1.

Preferred peptides according to the invention are also:

Suc-ENYLRLIEELIRKAQEQQEKNEAALRGSGC(cholesteryl-
oxycarbonylmethyl)-NH₂
Suc-ENYLRLIEELIRKAQEQQEKNEAALRGSGK(palmitoyl)-NH₂

Furthermore the peptides according to the present invention, preferably in a pharmaceutical composition are, or can be, used for the inhibition of the HIV fusion with, or HIV entry in, a host cell.

Those pharmaceutical compositions comprise the inventive peptide(s) together with a pharmaceutically acceptable carrier.

### DEFINITIONS

By the term “amino acid” is meant, for purposes of the specification and claims and in reference to the peptides according to the present invention, to refer to a molecule that has at least one free amine group and at least one free carboxyl group and may further comprise one or more free chemical reactive groups other than an amine or a carboxyl group (e.g., a hydroxyl, a sulfhydryl, etc). The amino acid may be a naturally occurring L-amino acid (depicted in this specification as a capital letter in the sequence), or its corresponding D-enantiomer (depicted in this specification as a small letter in the sequence), a (synthetic) non-naturally occurring amino acid (e.g. represented with the 3-letter code in the sequence such as Tba and the like), a modified amino acid, an amino acid derivative, an amino acid precursor, and/or a conservative substitution. A person skilled in the art would know that the choice of amino acids incorporated into a peptide will depend, in part, on the specific physical, chemical or biological characteristics required of the antiviral peptide. Such characteristics are determined, in part, by determination of helicity and antiviral activity. For example, a skilled person would know that amino acids in a synthetic peptide may be comprised of one or more of a naturally occurring (L-) amino acid and its corresponding D-enantiomer, or a non-naturally occurring amino acid like Tba and the like.

A “conservative substitution” is used in this specification to mean one or more amino acids substitution in the sequence of the synthetic peptide such that the synthetic peptide still demonstrate the unexpected, improved biological activity. This includes substitutions of amino acids having substantially the same charge, size, hydrophilicity, and/or aromaticity as the amino acid replaced.

A “CLIPS” peptide is a peptide according to the invention which comprises a peptide structure at the N-terminal part resulting from the linkage of the first to the fifth amino acid (wherein a free thiol function is needed) at said N-terminal part via one of the B1, B2, B3, B21 or B22 moieties. A method to obtain such CLIPS peptides is described in WO 2004/077062.

The term “HIV” refers to Human Immunodeficiency Virus, and more preferably HIV-1.

A “pharmaceutically acceptable carrier” means a carrier medium that does not significantly alter the biological activity of the peptide according to the invention to which it is added. Such carriers are for instance (buffered) water, isotonic aqueous buffer solutions, aqueous alcohol and the like.

The term “linker” refers to a compound or moiety that acts as a molecular bridge to operably link two different molecules (e.g. wherein one portion of the linker binds to a peptide according to the invention and wherein another portion of the linker binds to cholesterol or a derivative thereof)

“EC₅₀” (=half maximal effective concentration) is a measure of the effectiveness of a compound in inhibiting a biological function. This quantitative measure indicates how much of a particular drug or other substance (inhibitor) is needed to inhibit a given biological process (or component of a process, i.e. an enzyme, cell, cell receptor or microorganism) by half. According to the FDA, EC₅₀ represents the concentration of a drug that is required for 50% inhibition in vitro

Nomenclature Used in this Specification

For the L-natural amino acids, as known in the art, the following abbreviations were used:

Symbol

| Name | 3-Letter | 1-Letter |
|---|---|---|
| Alanine | Ala | A |
| Arginine | Arg | R |
| Asparagine | Asn | N |
| Aspartic acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

For the non-natural amino acids the following nomenclature (3-letter code) is used:

| Name | 3-Letter code | Amino acid structure |
|---|---|---|
| L-2-Amino-butyric acid | Abu | 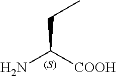 |
| L-3-Cyclohexyl-Alanine | Cha | 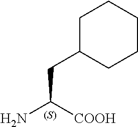 |
| L-Homo-Cysteine | Hcy | 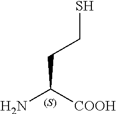 |
| L-Norvaline | Nva | 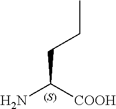 |
| L-3-tButyl-Alanine | Tba | 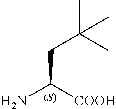 |

For those peptides according to the invention wherein the first amino acid and the fifth amino acid is either C or Hcy, said amino acids are connected by B1, B2, B3, B21 or B22 and the explanation for these abbreviations is clarified hereunder:

| CLIPS-Name | CLIPS flag | CLIPS structure |
|---|---|---|
| para-xylene | B1 | 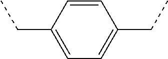 |
| meta-xylene | B2 | 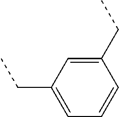 |
| ortho-xylene | B3 | 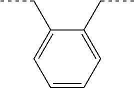 |
| 2,7-dimethylnaphtyl | B21 | 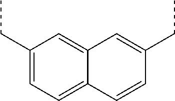 |
| 1,4-dimethylnaphtyl | B22 | 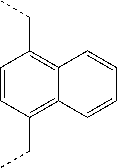 |

For the capping groups used of the peptides according to the invention, the following abbreviations are used and explained hereunder:

| N-Capping | Name | Capping Structure |
|---|---|---|
| Ac | Acetyl | 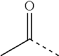 |
| Butanoyl | Butanoyl | 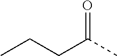 |
| Isovaleryl | Isovaleryl | 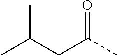 |
| Pentanoyl | Pentanoyl | 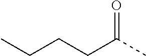 |
| Hexanoyl | Hexanoyl | 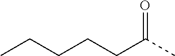 |
| Palmitoyl | Palmitoyl | 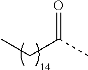 |
| Suc | Succinyl | 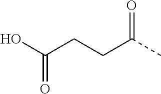 |
| Bzl | Benzyl | 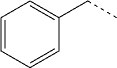 |

Peptides according to the invention are listed in the Table below:

For sake of clarity: the numbering, in the Table below, “636-661” corresponds to the amino acid numbering in gp160 of HIV wherein position number 637 is considered the first named amino acid in the peptides according to the invention. So for instance position number 646 is the tenth named amino acid and position number 660 is considered the named twenty-fourth amino acid in the peptides according to the invention accordingly.

In addition it is clarified that any amino acid sequence in the Table below starts with the respective N-capping group at the left end side and ends at the right end side with a carboxamide.

| N-Capping | 636 | 637 | 638 | 639 | 340 | 641 | 642 | 643 | 644 |
|---|---|---|---|---|---|---|---|---|---|
| Suc | E | C | Y | L | A | C | I | E | A |
| Ac | A | Hcy | | Nva | R | Hcy | | D | E |
| Butanoyl | a | C(Bzl) | | Abu | | L | | | |
| Isovaleryl | — | N | | I | | | | | |
| Pentanoyl | | | | F | | | | | |
| Hexanoyl | | | | Tba | | | | | |
| | | | | V | | | | | |
| | | | | W | | | | | |
| | | | | Cha | | | | | |
| | | | | A | | | | | |
| | | | | lipophilic | | | | acidic | alanine or |
| | | | | amino | | | | amino | a acidic |
| | | | | acid | | | | acid | amino acid |

| N-Capping | 645 | 646 | 647 | 648 | 649 | 650 | 651 | 652 |
|---|---|---|---|---|---|---|---|---|
| Suc | L | I | R | A | A | Q | E | Q |
| Ac | | Tba | K | K | Nva | R | | |
| Butanoyl | | | | R | Abu | | | |
| Isovaleryl | | | | | | | | |
| Pentanoyl | | | | | | | | |
| Hexanoyl | | | | | | | | |
| | | lipophilic | a basic | alanine or | lipophilic | | | |
| | | amino | amino | a acidic | amino | | | |
| | | acid | acid | amino acid | acid | | | |

| N-Capping | 653 | 654 | 655 | 656 | 657 | 658 | 659 | 660 | 661 | CLIPS |
|---|---|---|---|---|---|---|---|---|---|---|
| Suc | Q | E | K | N | E | A | A | L | R | B1 |
| Ac | | | | | | | L | | r | B2 |
| Butanoyl | | | | | | | | | L | B3 |
| Isovaleryl | | | | | | | | | Tba | B21 |
| Pentanoyl | | | | | | | | | K(palmitoyl) | B22 |
| Hexanoyl | | | | | | | | | — | |
| Hexanoyl | | | | | | | | | | |
| | | | | | | | lipophilic | | | |
| | | | | | | | amino | | | |
| | | | | | | | acid | | | |

The preferred seven (7) peptides according to the invention are listed below:

Pentanoyl-E-C-Y-L-A-C-I-E-A-L-I-R-A-A-
Q-E-Q-Q-E-K-N-E-A-A-L-R-NH₂

Connection of the first called amino acid (C) to the fifth called amino acid (C) is via B1.

Pentanoyl-E-C-Y-L-A-C-I-E-E-L-I-R-K-A-
Q-E-Q-Q-E-K-N-E-A-A-L-R-NH₂

Connection of the first called amino acid (C) to the fifth called amino acid (C) is via B1.

Isovaleryl-E-C(Bzl)-Y-L-A-L-I-E-E-L-I-R-
K-A-Q-E-Q-Q-E-K-N-E-A-A-L-R-NH₂
Suc-E-C-Y-L-R-C-I-E-E-L-I-R-K-A-Q-E-Q-Q-
E-K-N-E-A-A-L-R-
GSGC(cholesteryloxycarbonylmethyl)-NH₂

Connection of the first called amino acid (C) to the fifth called amino acid (C) is via B1.

Suc-E-C-Y-L-R-C-I-E-E-L-I-R-K-A-Q-E-Q-Q-
E-K-N-E-A-A-L-R-GSGK(palmitoyl)-NH₂

Connection of the first called amino acid (C) to the fifth called amino acid (C) is via B1.

Suc-E-N-Y-L-R-L-I-E-E-L-I-R-K-A-Q-E-Q-Q-
E-K-N-E-A-A-L-R-
GSGC(cholesteryloxycarbonylmethyl)-NH₂
Suc-E-N-Y-L-R-L-I-E-E-L-I-R-K-A-Q-E-Q-Q-
E-K-N-E-A-A-L-R-GSGK(palmitoyl)-NH₂

Most preferred are the two following peptides from the listing above:

Suc-E-N-Y-L-R-L-I-E-E-L-I-R-K-A-Q-E-Q-
Q-E-K-N-E-A-A-L-R-
GSGC(cholesteryloxycarbonylmethyl)-NH₂
Suc-E-N-Y-L-R-L-I-E-E-L-I-R-K-A-Q-E-Q-
Q-E-K-N-E-A-A-L-R-GSGK(palmitoyl)-NH₂

### Preparation of the Peptides According to the Invention.

### General Procedure for Fmoc-Synthesis of Peptides:

Peptides with a C-terminal carboxamide were synthesized by Fmoc-chemistry on solid-phase using 4-(2′,4′-dimethoxyphenyl-Fmoc-aminomethyl)-phenoxy (RinkAmide) resin. Side-chain functionalities were protected as N-Boc (K,W), O-t-Bu (D,E,S,T,Y), N-Trt (H,N,Q), S-Trt (C, Hcy), S-Acm (C) or N-Pbf (R,r) groups. (Acm: Acetamidomethyl, Boc: tert. Butoxycarbonyl, t-Bu: tert. Butyl, Fmoc: 9-Fluorenylmethoxycarbonyl, Pbf: 2,2,4,6,7-Pentamethyldihydro-benzofuran-5-sulfonyl, Trt: trityl)

A coupling protocol, using a 5-fold excess of HBTU (2-(1H-Benzotriazole-1-yl)-1,1,3,3-tetramethyluronium hexafluorophosphate)/HOBt (Hydroxybenzo-triazole)/Fmoc-amino acid/DIEA (N,N-diisopropylethylamine) (1:1:1:2) in NMP (N-methyl-2-pyrrolidone) with a 20-30 minute activation time using double couplings, was employed for every amino acid coupling step. Acetylation of the peptide was performed by reacting the resin with Ac₂O (acetic anhydride)/DIEA in NMP (1:0.1:10, v/v/v) for 30 min at room temperature. Succinylation was performed by reacting the peptide-resin with 10 eq. of succinic anhydride and 2 eq. of DIEA in NMP. For the N-terminal capping with butanoyl, isovaleryl, pentanoyl and hexanoyl, the same protocol as for the amino acid coupling was used.

The peptides were cleaved from the resin and completely deprotected by reaction with TFA (trifluoroacetic acid, 40 mL/mmol resin) containing 13.3% (w) phenol, 5% (v) thioanisole, 2.5% (v) 1,2-ethanedithiol, and 5% (v) milliQ-H₂O for 2-3 hours at room temperature. Precipitation and washing with ice-cold diethyl ether/pentane (1:1) followed by lyophilization of the precipitated material from ACN (acetonitrile)/water (1:1) afforded the crude peptide which was purified by reversed-phase high performance liquid chromatography (RP-HPLC).

### Preparative Peptide Purification by Reversed-Phase HPLC:

Peptide purification was carried out using a Waters RCM module equipped with Delta-Pak cartridges (25×100 or 40×210 mm, 15 μm, C18-100 Å, Waters, USA) in a linear AB gradient of 1% B/min (solvent A: 0.05% TFA in water, solvent B: 0.05% TFA in ACN) at a flow rate of 40 or 100 mL/min (the starting percentage of the gradient was based on the retention time in analytical HPLC). Peptide detection was done at 215 nm. Pure fractions were collected and lyophilized, yielding the trifluoroacetate salt of the peptide.

CLIPS Reaction with Peptides:

An example of a so-called CLIPS reaction (exemplified in FIG. 1) was performed by reacting the completely unprotected peptide at a concentration of 0.5 mM in a mixture of ACN and water (1:3), with 1.25 eq. of CLIPS reagent (α,α′-dibromo-o-xylene, α,α′-dibromo-m-xylene, α,α′-dibromo-p-xylene, 1,4-bis-(bromomethyl)-naphtalene, 2,7-bis-(bromomethyl)-naphtalene, or benzylbromide), the pH of the reaction mixture was adjusted to 7-8 by the addition of an aqueous 0.2 M ammonium bicarbonate solution. After one hour, the reaction mixture was quenched with 10% TFA. ACN was partially removed (rotary evaporator) before purification; in case of hydrophobic peptides no ACN was removed.

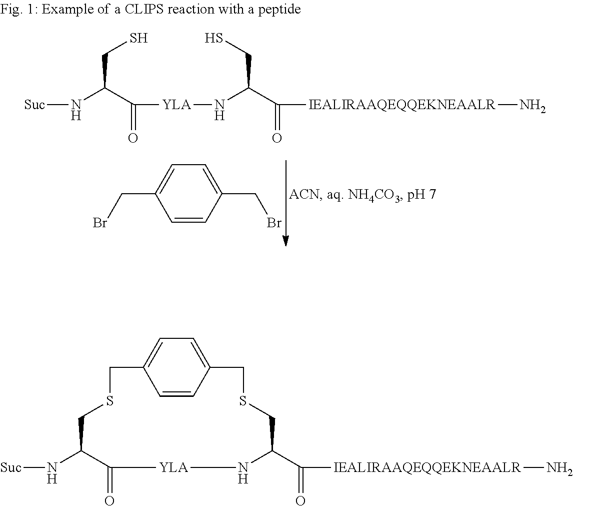

### Synthesis of Cholesterol Linked Peptide 82 as Example:

Peptide Intermediate I-1 was synthesized on solid phase (250 μmol+6×100 μmol) using the general synthesis protocol as described above. The C-terminal cysteine was coupled as Fmoc-Cys(Acm)-OH (Orpegen Peptide Chemicals GmbH, Germany). The crude peptide (2793 mg) was purified in four batches on a Waters RCM module equipped with Delta-Pak cartridges (40×210 mm, 15 μm, C18-100 Å, Waters, USA) in a linear gradient starting from 22% to 42% B in 20 minutes (solvent A: 0.05% TFA in water, solvent B 0.05% TFA in ACN) at a flow rate of 100 mL/min. Pure fractions were collected and concentrated under reduced pressure (rotary evaporator), 820 mg of the trifluoroacetate salt of I-1 was obtained after lyophilization from ACN/water (1.1).

Peptide Intermediate I-1 (820 mg, 225 μmol) was dissolved in mixture of water (110 mL) and ACN (340 mL), α,α′-dibromo-p-xylene (74 mg, 280 μmol) in ACN (28 mL) was added, followed by the addition of an aqueous ammonium bicarbonate solution (56 mL of 0.2 M solution). The reaction mixture was stirred for one hour, acidified with 10% TFA to pH 3 and directly purified on a Davisil C18 preparative HPLC column (50×277 mm, 16-24 μm, 150 Å, Grace, USA) in a linear gradient of 23% to 43% B in 20 minutes (solvent A: 0.05% TFA in water, solvent B: 0.05% TFA in ACN) at a flow rate of 120 mL/min. The injection was run for 5 min at 60 mL/min in 13% B. After evaporation (rotary evaporator) and lyophilization (from ACN/water (1:1)), 576 mg of the trifluoroactate salt of I-2 was obtained.

Peptide Intermediate I-2 (576 mg, 154 μmol) was dissolved in an aqueous 8 M guanidinium hydrochloride solution (15.4 mL), followed by the addition of methanol (123 mL) and I₂ (15.4 mL of a 34 mg/mL solution in methanol, 2 mmol) under vigorous stirring. After 15 min, DTT (dithiothreitol, 7.7 mL) was added and the pH of the reaction mixture was adjusted to pH using 38.5 mL of an aqueous 0.2 M ammonium bicarbonate solution. Methanol was evaporated under reduced pressure (rotary evaporator) and the obtained crude product was purified on a Davisil C18 preparative HPLC column (50×277 mm, 16-24 μm, 150 Å, Grace, USA), in a linear gradient of 24% to 44% B in 20 minutes (solvent A: 0.05% TFA in water, solvent B: 0.05% TFA in ACN) at a flow rate of 120 mL/min. The injection was run for 5 min at 60 mL/min in 14% B. After evaporation (rotary evaporator) and lyophilization (from ACN/water (1:1)), 421 mg of peptide Intermediate I-3 was obtained.

Cholesterol (162 mg in 5 mL DCM (dichloromethane), 420 μmol), bromoacetic acid (55.6 mg in 2 mL DCM, 400 μmol) and DMAP (4-dimethylaminopyridine, 5 mg in 1 mL DCM, 40 μmol) were mixed under vigorous stirring. DCC (dicyclohexyl carbodiimide, 82.5 mg in 1 mL DCM, 400 μmol) was added and the reaction mixture was stirred for two hours at room temperature. The precipitate was removed by filtration. Half of the obtained filtrate was added to a solution of I-3 (420.1 mg, 0.115 mmol) in DMF (N,N-dimethylformamide, 10 mL), followed by the addition of a concentrated aqueous ammonium bicarbonate solution until a pH of 7 was obtained. The reaction mixture was stirred until complete conversion (±70 min, monitoring was done by LC-MS) and subsequently quenched by the addition of TFA (pH 3). Most DCM was evaporated by bubbling with nitrogen. The peptide was purified on a Waters RCM module equipped with Delta-Pak cartridges (40×210 mm, 15 μm, C18-100 Å, Waters, USA) in a linear gradient of 45% to 75% B in 30 minutes (solvent A: 0.05% TFA in water, solvent B: 0.05% TFA in ACN) at a flow rate of 100 mL/min. The injection was run for 5 min at 50 mL/min in 35% B. Pure fractions were concentrated under reduced pressure (rotary evaporator) and lyophilized from ACN/water (1:1), to yield 245 mg of the cholesterol linked peptide 82 as a trifluoroacetate salt.

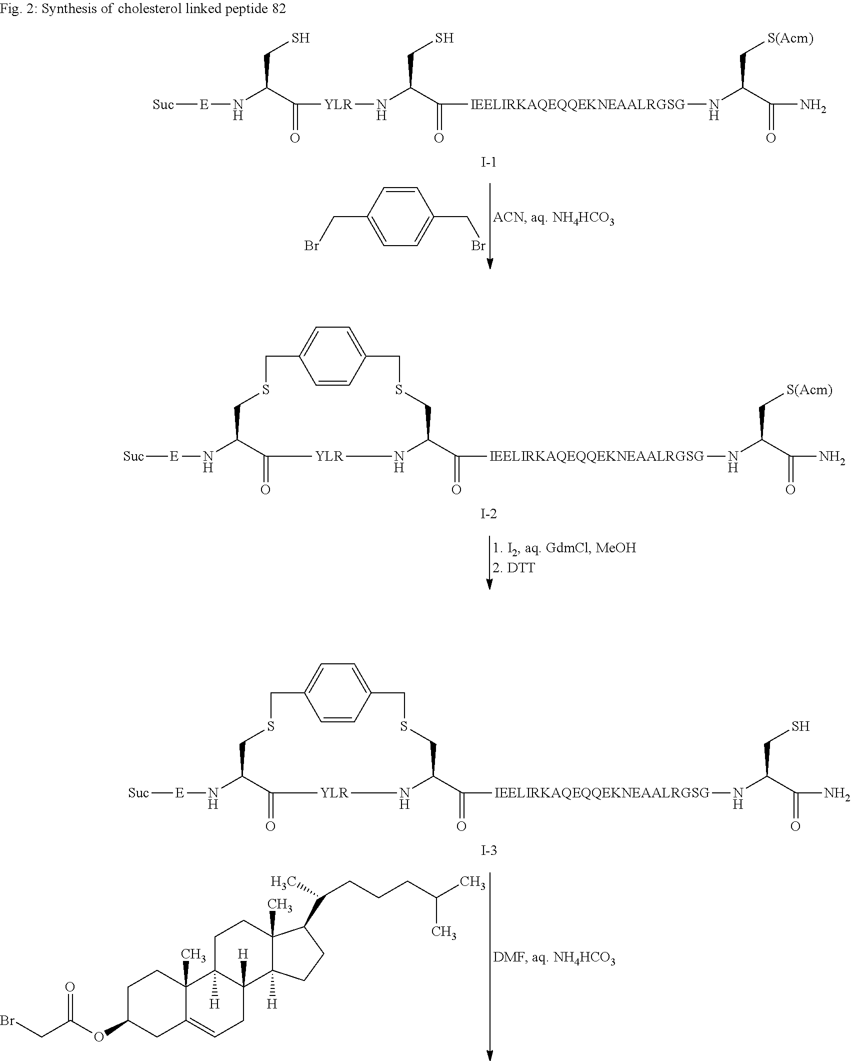

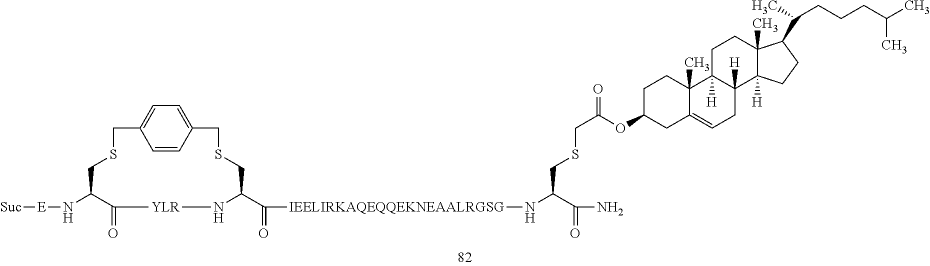

### UPLC Analysis:

The UPLC (Ultra Performance Liquid Chromatography) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's molecular weight (MW). Data acquisition was performed with appropriate software.

Peptides are described by their experimental retention time (Rt) and their molecular weight. Molecular weight was calculated from the experimental mass to charge (m/z) ratios from all the observed protonation states of a peptide using MassLynx software (Waters, USA).

Hereinafter, “BEH” bridged ethylsiloxane/silica hybrid, “DAD” Diode Array Detector, “Q-T of” Quadrupole Time-of-flight mass spectrometers, “SQD” Single Quadrupole Detector.

TABLE
UPLC Method codes (Flow expressed in mL/min; column temperature
(T) in ° C.).

| Method | | | | | |
|---|---|---|---|---|---|
| code | Instrument | Column | Mobile phase | Gradient | Flow/Col T |
| A | Waters: | Waters: | A: 0.05% TFA | 25% B/min | 1/50 |
| | Acquity ® | BEH130 C18 | in H₂O | starting at 5% B | |
| | UPLC ® - | (1.7 μm, | B: 0.05% TFA | | |
| | DAD and | 2.1 * 50 mm) | in CH₃CN | | |
| | SQD | | | | |
| B | Waters: | Waters : | A: 0.1% | From 5% B to 95% | 0.2/40 |
| | Acquity ® | BEH300 C18 | HCOOH + 5% | B in 14.00 min, | |
| | UPLC ® - | (1.7 μm , | CH₃OH in H₂O | hold for 1 min | |
| | DAD and | 2.1 * 150 m) | B: CH₃CN | | |
| | Q-TOF | | | | |

### Description of the Assays Used and Results:

Standard Anti-Viral-Experiment “AVE” (wild type HIV strain IIIB+HIV strain HXB2D side directed mutants V38A and Q40H)

### Assay Principles

The HIV-1 replication assay measures virus replication (HIV wild type virus strain 111B or HXB2D, or a HIV mutant virus strain HXB2D with mutation V38A or Q40H in the gp41 gene) as an induction of enhanced green fluorescent protein (EGFP) expression. The indicator MT4-LTR-EGFP cells contain an EGFP gene under the control of the HIV-1 LTR promoter sequence. Successful HIV-1 infection results in viral Tat protein expression and subsequent induction of EGFP expression. Compounds/peptides inhibiting HIV-1 infection are expected to reduce EGFP expression as compared to the untreated HIV-infected control.

### Methods

Serial 4-fold dilutions of test compounds/peptides were mixed with HIV-1 (IIIB, HXB2D, or a HXB2D mutant virus (V38A or Q40H)) and MT4-LTR-EGFP cells and incubated at 37° C. After 3 days, the wells were examined for EGFP expression using an argon laser-scanning microscope. The effective compound/peptide concentration inhibiting 50% of the virus-induced EGFP signal (EC₅₀) was determined by linear interpolation of the EGFP signal vs. logarithm of the compound concentration; (T20, C34 and Sifuvirtide were added as reference compounds). For the V38A and Q40H mutant viruses, results were reported as a fold change in EC₅₀, as compared with a drug-susceptible wild type strain HXB2D, which forms the backbone of the mutant virus.
Standard AVE (50% Human Serum)

### Assay Principles

The HIV-1 replication assay (with 50% human serum) measures virus replication as an induction of enhanced green fluorescent protein (EGFP) expression, in the presence of 50% human serum. The indicator MT4-LTR-EGFP cells contain an EGFP gene under the control of the HIV-1 LTR promoter sequence. Successful HIV-1 infection results in viral Tat protein expression and subsequent induction of EGFP expression. Compounds/peptides inhibiting HIV-1 infection are expected to reduce EGFP expression as compared to the untreated HIV-infected control. Compounds/peptides binding to human serum are expected to have a reduced activity for inhibiting the virus in the assay.

### Methods

Serial 4-fold dilutions of test compounds/peptides were mixed with HIV-1 and MT4-LTR-EGFP cells and incubated at 37° C., in the presence of 50% human serum. After 3 days, the wells were examined for EGFP expression using an argon laser-scanning microscope. The effective compound/peptide concentration inhibiting 50% of the virus-induced EGFP signal (EC₅₀) was determined by linear interpolation of the EGFP signal vs. logarithm of the compound concentration; T20, C34 and Sifuvirtide were added as reference compounds. Results were reported as a fold change in EC₅₀, as compared with the acquired EC₅₀ in the assay without human serum.

| | | | Calculated | Measured |
|---|---|---|---|---|
| | Sequence | CLIPS | MW | MW |
| T20 | AC-YTSLIHSLIEESQNQQEKNEQELLELDKWASLWNWF-NH₂ | — | 4492.0 | 4491.3 |
| C34 | Ac-WMEWDREINNYTSLIHSLIEESQNQQEKNEQELL-NH₂ | — | 4289.6 | 4289.3 |
| C34-PBD* | Ac-NNYTSLIHSLIEESQNQQEKNEQELL-NH₂ | — | 3143.4 | 3142.9 |
| | (PBD truncated C34) | | | |
| Sifuvirtide | Ac-SWETWEREIENYTRQIYRILEESQEQQDRNERDLLE-NH₂ | — | 4727.1 | 4726.9 |
| Sifuvlrtlde- | Ac-ENYTRQIYRILEESQEQQDRNERDLL-NH₂ | — | 3380.6 | 3380.4 |
| PBD | (PBD truncated Sifuvirtide) | | | |
| 1 | Suc-ANYLALIEALIRAAQEQQEKNEAAL-NH₂ | — | 2870.2 | 2869.1 |
| 2 | Ac-ANYAALIEALIRAAQEQQEKNEAALR-NH₂ | — | 2926.3 | 2926.8 |
| 3 | Suc-ANYAALIEALIRAAQEQQEKNEAALR-NH₂ | — | 2984.3 | 2985.2 |
| 4 | Ac-A-C(Bzl)-YAALIEALIRAAQEQQEKNEAALR-NH₂ | — | 3005.5 | 3006 |
| 5 | Ac-ANYAALIEALIRKAQEQQEKNEAALR-NH₂ | — | 2983.4 | 2983.2 |
| 6 | Ac-ANYLALIEALIRAAQEQQEKNEAALR-NH₂ | — | 2968.4 | 2968.1 |
| 7 | Suc-ANYLALIEALIRAAQEQQEKNEAALR-NH₂ | — | 3026.4 | 3026.1 |
| 8 | Ac-ENYLALIEALIRAAQEQQEKNEAALR-NH₂ | — | 3026.4 | 3028.2 |
| 9 | Ac-ENYLALIEALIRAAQEQQEKNEAALL-NH₂ | — | 2983.4 | 2982.8 |
| 10 | Ac-ANYLALIEELIRAAQEQQEKNEAALR-NH₂ | — | 3326.4 | 3027.7 |
| 11 | Suc-ANYLALIEAL-Tba-RAAQEQQEKNEAALR-NH₂ | — | 3040.5 | 3041.8 |
| 12 | Suc-aNYLALIEALIRAAQEQQEKNEAALr-NH₂ | — | 3026.4 | 3025.9 |
| 13 | Suc-ANYLALIEALIRAAQEQQEKNEAALK | — | 3236.8 | 3238.4 |
| | (palmitoyl)-NH₂ | | | |
| 14 | Pentanoyl-ANYAALIEALIRAAQEQQEKNEAALR-NH₂ | — | 2968.4 | 2968 |
| 15 | Pentanoyl-ANYLALIEALIRAAQEQQEKNEAALR-NH₂ | — | 3010.5 | 3010 |
| 16 | Pentanoyl-ANYAALIEALIRKAQEQQEKNEAALR-NH₂ | — | 3025.5 | 3025.3 |
| 17 | Isovaleryl-ANYAALIEALIRAAQEQQEKNEAALR-NH₂ | — | 2968.4 | 2967.9 |
| 18 | Isovaleryl-E-C(Bzl)- | — | 3262.8 | 3263 |
| | YLALIEELIRKAQEQQEKNEAALR-NH₂ | | | |
| 19 | Suc-ENYLALIEELIRKAQEQQEKNEAALR-NH₂ | — | 3199.6 | 3200.1 |
| 20 | Suc-ENYLRLIEELIRKAQEQQEKNEAALR-NH₂ | — | 3284.7 | 3284.3 |
| 21 | Ac-CYAACIEALIRAAQEQQEKNEAALR-NH₂ | B1 | 2936.4 | 2937.2 |
| 22 | Ac-CYAACIEALIRAAQEQQEKNEAALR-NH₂ | B21 | 2986.5 | 2986.9 |
| 23 | Suc-CYLACIEELIRKAQEQQEKNEAAL-Tba-NH₂ | B2 | 3122.6 | 3123.3 |
| 24 | Suc-CYLACIEELIRKAQEQQEKNEAAL-Tba-NH₂ | B1 | 3122.6 | 3121.8 |
| 25 | Suc-CYLACIEELIRKAGEQQEKNEAAL-Tba-NH₂ | B21 | 3172.7 | 3173.2 |
| 26 | SUC-CYLACIEALIRAAQEQQEKNEAALR-NH₂ | B1 | 3036.5 | 3036.6 |
| 27 | Suc-CYLACIEELIRKAQEQQEKNEAAL-Tba-NH₂ | B3 | 3122.6 | 3123.8 |
| 28 | Suc-Hcy-YLA-Hcy-IEALIRAAREQQEKNEALLR-NH₂ | B1 | 3134.7 | 3134.9 |
| 29 | Suc-ECYLRCIEELIRKAQEQQEKNEAALR-NH₂ | B1 | 3365.9 | 3365.9 |
| 30 | SUC-ACYLACIDELIKKAQEQQEKNEAALR-NH₂ | B1 | 3180.7 | 3181 |
| 31 | Suc-ACYLAC)EELIRKAQEQQEKNEAALR-NH₂ | B1 | 3222.7 | 3223 |
| 32 | Suc-ENYLRLIEELIRKAQEQQEKNEAALRGSGC | — | 4015.7 | 4015.3 |
| | (cholesteryl-oxycarbonylmethyl)-NH₂ | | | |
| 33 | SUC-ECYLACIEALIRAAQEQQEKNEAALR-NH₂ | B1 | 3165.6 | 3166.9 |
| 34 | Suc-ACYLACIEELIRKAQEQQEKNEAALr-NH₂ | B1 | 3222.7 | 3222.9 |
| 35 | Suc-ANYLALIEALIRAAQEQQEKNEAALRGSGC | - | 3757.4 | 3757 |
| | (cholesteryl-oxycarbonylmethyl)-NH₂ | | | |
| 36 | SUC-ACYLACIDELIAKAQEQQEKNEAALR-NH₂ | B1 | 3123.6 | 3123.4 |
| 37 | Ac-ECYLACIEELIRKAQEQQEKNEAALR-NH₂ | B1 | 3222.7 | 3222.9 |
| 38 | Pentanoyl-ECYLACIEALIRAAQEQQEKNEAALR-NH₂ | B1 | 3149.7 | 3149.6 |
| 39 | Penlanoyl-ECYLACIEALIRAAQEQQEKNEAALR-NH₂ | B21 | 3199.7 | 3199.9 |
| 40 | Butanoyl-ECYLACIEELIRKAQEQQEKNEAALR-NH₂ | B1 | 3250.8 | 3251.8 |
| 41 | Pentanoyl-ECYLACIEELIRKAQEQQEKNEAALR-NH₂ | B1 | 3264.8 | 3264.9 |
| 42 | Hexanoyl-ECYLACIEELIRKAQEQQEKNEAALR-NH₂ | B1 | 3278.8 | 3279.9 |
| 43 | Suc-ACYLACIEALIRAAQEQQEKNEAALR-NH₂ | B1 | 3107.6 | 3107,8 |
| 44 | Suc-ACY-Nva-ACIEALIRAAQEQQEKNEAALR-NH₂ | B1 | 3093.6 | 3094.7 |
| 45 | AC-ECYLACIEALIRAAQEQQEKNEAALR-NH₂ | B1 | 3107.6 | 3108.5 |
| 46 | Suc-ACYLACIEALIRA-Nva-QEQQEKNEAALR-NH₂ | B1 | 3135.6 | 3136.1 |
| 47 | Suc-ACYLACIEALIRA-Abu-QEQQEKNEAALR-NH₂ | B1 | 3121.6 | 3121.6 |
| 48 | Ac-ACYLACIEALIRKAQEQQEKNEAALR-NH₂ | B1 | 3106.6 | 3107.5 |
| 49 | Suc-ACY-Abu-ACIEALIRAAQEQQEKNEAALR-NH₂ | B1 | 3079.5 | 3080.6 |
| 50 | Ac-ECYLRCIEALIRAAQEQQEKNEAALR-NH₂ | B1 | 3192.7 | 3193.4 |
| 51 | AC-ACYLACIEALIRRAQEQQEKNEAALR-NH₂ | B1 | 3134.7 | 3135.3 |
| 52 | Pentanoyl-ENYLALIEELIRKAQEQQEKNEAALR-NH₂ | — | 3183.6 | 3183.3 |
| 53 | Ac-ECYAACIEELIRKAQEQQEKNEAALR-NH₂ | B1 | 3180.6 | 3181.8 |
| 54 | Ac-ACYLACIEALIRAAQEQQEKNEAALL-NH₂ | B1 | 3006.5 | 3007.4 |
| 55 | Suc-ENYLRLIEELIRKAQEQQEKNEAALRGSGK | — | 3852.4 | 3851.6 |
| | (palmitoyl)-NH₂ | | | |
| 56 | Ac-ACYLACIEALIRAAQEQQEKNEAALR-NH₂ | B1 | 3049.6 | 3050.1 |
| 57 | Ac-ACYIACIEALIRAAQEQQEKNEAALR-NH₂ | B1 | 3049.6 | 3049.6 |
| 58 | Ac-ACYIACIEALIRAAQEQQEKNEAALR-NH₂ | B21 | 3099.6 | 3100.8 |
| 59 | Ac-ACY-Cha-ACIEALIRAAQEQQEKNEAALR-NH₂ | B1 | 3089.6 | 3090.6 |
| 60 | Ac-ACY-Tba-ACIEALIRAAQEQQEKNEAALR-NH₂ | B1 | 3063.6 | 3063.3 |
| 61 | Ac-ACYFACIEALIRAAQEQQEKNEAALR-NH₂ | B1 | 3083.6 | 3083.9 |
| 62 | Ac-ACYVACIEALIRAAQEQQEKNEAALR-NH₂ | B1 | 3035.5 | 3035.9 |
| 63 | AC-ACYWACIEALIRAAQEQQEKNEAALR-NH₂ | B1 | 3122.6 | 3123.2 |
| 64 | Ac-ACYAACIEALIRAAQEQQEKNEAALR-NH₂ | B3 | 3007.5 | 3008.8 |
| 65 | Ac-ACYAACIEALIRAAQEQQEKNEAALR-NH₂ | B2 | 3007.5 | 3008.9 |
| 66 | Ac-ACYAACIEALIRAAQEQQEKNEAALR-NH₂ | B21 | 3057.5 | 3058.7 |
| 67 | Suc-ACYAACIEALIRAAQEQQEKNEAALR-NH₂ | B1 | 3065.5 | 3066.9 |
| 68 | Ac-ACYAACIEALIRAAQEQQEKNEAALR-NH₂ | B1 | 3007.4 | 3007.6 |
| 69 | Ac-ANYLALIEALIRAAQEQQEKNEAALRGSGK | — | 3766.4 | 3765.6 |
| | (cholesteryl-succinyl)-NH₂ | | | |
| 70 | Ac-ACYAACIDALIRAAQEQQEKNEAALR-NH₂ | B1 | 2993.4 | 2994.0 |
| 71 | Ac-ACYAACIEALIRKAQEQQEKNEAALR-NH₂ | Bl | 3064.6 | 3065.2 |
| 72 | Ac-ACYAACIEELIRKAQEQQEKNEAALR-NH₂ | B1 | 3122.6 | 3123.1 |
| 73 | Ac-ACYAACIEALIRAAQEQQEKNEALLR-NH₂ | B1 | 3049.5 | 3050.3 |
| 74 | AC-ECYAACIEALIRAAQEQQEKNEAALR-NH₂ | B1 | 3065.5 | 3065.5 |
| 75 | Ac-aCYAACIEALIRAAQEQQEKNEAALr-NH₂ | B21 | 3007.5 | 3007.9 |
| 76 | Ac-aCYAACIEALIRAAQEQQEKNEAALr-NH₂ | B21 | 3057.5 | 3057.7 |
| 77 | Ac-A-Hcy-YAA-Hcy-IEALIRAAQEQQEKNEAALR-NH₂ | B1 | 3035.5 | 3036.3 |
| 78 | Ac-A-Hcy-YAA-Hcy-IEALIRAAQEQQEKNEAALR-NH₂ | B21 | 3085.6 | 3086.6 |
| 79 | Ac-A-Hcy-YAA-Hcy-IEALIRKAQEQQEKNEAALR-NH₂ | B21 | 3092.6 | 3093.3 |
| 80 | Ac-A-Hcy-YAA-Hcy-IEALIRKAQEQQEKNEAALR-NH₂ | B21 | 3142.7 | 3142.6 |
| 81 | Ac-ACYAACIEALIRAAQEQQEKNEAALRGSGC | B1 | 3738.4 | 3739.3 |
| | (cholesteryl-oxycarbonylmethyl)-NH₂ | | | |
| 82 | Suc-ECYLRCIEELIRKAQEQQEKNEAALRGSGC | B1 | 4096.9 | 4098.3 |
| | (cholesteryl-oxycarbonylmethyl)-NH₂ | | | |
| 83 | Ac-ACYAAcIEALIRAAQEQQEKNEAALR-NH₂ | B1 | 3007.5 | 3007.7 |
| 84 | AC-ACYAACIEALIRAAREQQEKNEAALR-NH₂ | B1 | 3035.5 | 3035.9 |
| 85 | Butanoyl-ECYLACIEELIRKAQEQQEKNEAALR-NH₂ | B1 | 3250.8 | 3251.8 |
| 86 | Hexanoyl-ECYLACIEELIRKAQEQQEKNEAALR-NH₂ | B1 | 3278.8 | 3279.9 |
| 87 | Ac-ACYAACIEALIRAAQEQQEKNEAALRGSGC | B1 | 3738.4 | 3739.3 |
| | (cholesteryl-oxycarbonylmethyl)-NH₂ | | | |
| 88 | Suc-ECYLRCIEELIRKAQEQQEKNEAALRGSGK | B1 | 3933.6 | 3933.4 |
| | (palmitoyl)-NH₂ | | | |
| 89 | AC-ACYAACIEALIRAAQEQQEKNEAALR-NH₂ | B22 | 3057.5 | 3058.3 |
| 90 | Suc-ECYLACIEELIRKAQEQQEKNEAALR-NH₂ | B1 | 3280.8 | 3279.9 |
| 91 | Pentanoyl-ECYLRCIEELIRKAQEQQEKNEAALR-NH₂ | B1 | 3349.9 | 3350.6 |
| 92 | Pentanoyl-ECYLCLIEELIRKAQEQQEKNEAALR-NH₂ | B1 | 3306.9 | 3307.1 |

| | | | HIV-IIIB + | HIV-HXB2D | HIV-HXB2D |
|---|---|---|---|---|---|
| | | | 50% HS (Fold | V38A mutant | Q40H mutant |
| | Rt | HIV-IIIB | change in | (fold change | (fold change |
| | (min)^{#} | (EC₅₀, nm) | EC₅₀) | in EC₅₀) | in EC₅₀) |
| T20 | 8.31 (B) | 17 | 3.9 | 75.8 | 49.7 |
| C34 | 7.80 (B) | 266 | 0.8 | 2.2 | 8.8 |
| C34-PBD* | 6.60 (B) | >49180 | ND | ND | ND |
| Sifuvirtide | 7.53 (B) | 2 | 1 | 1 | 2 |
| Slfuvlrtlde- | 5.96 (B) | 36400 | 0.8 | >3 | >3 |
| PBD | | | | | |
| 1 | 8.12 (B) | 4 | 0.4 | 57 | ND |
| 2 | 1.65 (A) | 77 | 1 | 61 | >28 |
| 3 | 1.64 (A) | 22 | 0.4 | 74 | ND |
| 4 | 1.86 (A) | 10 | 2 | 74 | ND |
| 5 | 6.8 (B) | 48 | 0.5 | 53 | ND |
| 6 | 8.07 (B) | 5 | 0.7 | 51 | ND |
| 7 | 7.77 (B) | 2 | 0.6 | 33 | 764 |
| 8 | 1.81 (A) | 2 | 0.4 | 46 | 900 |
| 9 | 8.59 (B) | 3 | 0.4 | 72 | 1196 |
| 10 | 1.83 (A) | 4 | 0.4 | 50 | >695 |
| 11 | 1.82 (A) | 1 | 0.8 | 26 | 368 |
| 12 | 7.79 (B) | 10 | 0.4 | 51 | ND |
| 13 | 2.60 (A) | 0.4 | 9 | 2 | 9 |
| 14 | 8.11 (B) | 13 | 2 | 46 | >67 |
| 15 | 8.75 (B) | 2 | 2 | 18 | 168 |
| 16 | 7.4 (B) | 8 | 0.8 | 49 | >108 |
| 17 | 8.07 (B) | 24 | 0.9 | 75 | >37 |
| 18 | 1.93 (A) | 1 | 3 | 3 | 53 |
| 19 | 1.55 (A) | 1 | ND | 22 | 151 |
| 20 | 6.80 (B) | 0.7 | 1 | 8 | 80 |
| 21 | 1.67 (A) | 5 | 3 | 75 | ND |
| 22 | 1.79 (A) | 35 | 2 | 19 | ND |
| 23 | 1.72 (A) | 1 | 0.6 | 45 | 807 |
| 24 | 1.73 (A) | 1 | 1 | 26 | 321 |
| 25 | 1.77 (A) | 0.6 | 3 | 31 | 352 |
| 26 | 1.78 (A) | 1 | 1 | 19 | 311 |
| 27 | 1.71 (A) | 3 | 0.7 | 28 | 307 |
| 28 | 1.85 (A) | 0.6 | 4 | 4 | 25 |
| 29 | 1.54 (A) | 0.8 | 0.6 | 7 | 98 |
| 30 | 1.59 (A) | 1 | 0.5 | 8 | 145 |
| 31 | 1.63 (A) | 1 | 0.4 | 14 | 238 |
| 32 | 2.28 (A*) | 0.04 | 13 | 1 | 1 |
| 33 | 1.74 (A) | 1 | 0.6 | 11 | 269 |
| 34 | 1.64 (A) | 2 | 0.3 | 19 | 265 |
| 35 | 2.55 (A*) | 0.3 | 4 | 4 | 24 |
| 36 | 1.65 (A) | 2 | 0.4 | 23 | 373 |
| 37 | 1.69 (A) | 2 | 0.7 | 7 | 108 |
| 38 | 2.01 (A) | 1 | 3 | 3 | 45 |
| 39 | 2.09 (A) | 7 | 5 | 2 | 11 |
| 40 | 1.86 (A) | 1 | 1 | 3 | 84 |
| 41 | 1.86 (A) | 1 | 2 | 2 | 42 |
| 42 | 1.98 (A) | 1 | 3 | 2 | 27 |
| 43 | 1.77 (A) | 2 | 1 | 19 | 103 |
| 44 | 1.79 (A) | 2 | 1 | 15 | 160 |
| 45 | 1.86 (A) | 2 | 1 | 14 | 64 |
| 46 | 1.92 (A) | 1 | 2 | 6 | 42 |
| 47 | 1.88 (A) | 1 | 2 | 17 | 261 |
| 48 | 1.74 (A) | 3 | 1 | 10 | 62 |
| 49 | 1.74 (A) | 6 | 0.6 | 33 | ND |
| 50 | 1.75 (A) | 2 | 2 | 12 | 33 |
| 51 | 1.72 (A) | 3 | 1 | 10 | 93 |
| 52 | 8.00 (B) | 1 | 2 | 5 | 38 |
| 53 | 1.55 (A) | 21 | 0.3 | 34 | >164 |
| 54 | 2.00 (A) | 3 | 2 | 31 | ND |
| 55 | 2.09 (A) | 0.05 | 3 | 1 | 1 |
| 56 | 1.86 (A) | 4 | 2 | 61 | ND |
| 57 | 1.90 (A) | 4 | 2 | 23 | ND |
| 58 | 1.97 (A) | 4 | 6 | | 23 |
| 59 | 2.01 (A) | 3 | 3 | 18 | 44 |
| 60 | 1.94 (A) | 5 | 3 | 18 | ND |
| 61 | 1.86 (A) | 4 | 4 | 20 | ND |
| 62 | 1.85 (A) | 10 | 2 | 15 | ND |
| 63 | 1.81 (A) | 7 | 3 | 22 | ND |
| 64 | 1.78 (A) | 56 | 0.8 | 15 | ND |
| 65 | 1.77 (A) | 17 | 1 | 16 | ND |
| 66 | 1.86 (A) | 3 | 2 | 66 | 231 |
| 67 | 1.70 (A) | 8 | 0.8 | 30 | ND |
| 68 | 1.77 (A) | 48 | 2 | 21 | >67 |
| 69 | 13.06 (B) | 0.3 | 18 | 1 | 11 |
| 70 | 1.68 (A) | 23 | 1 | 11 | ND |
| 71 | 1.58 (A) | 8 | 1 | 11 | ND |
| 72 | 1.58 (A) | 15 | 0.6 | 10 | ND |
| 73 | 1.78 (A) | 21 | 3 | 60 | ND |
| 74 | 1.70 (A) | 53 | 0.9 | 33 | ND |
| 75 | 1.76 (A) | 29 | 4 | 21 | ND |
| 76 | 1.85 (A) | 15 | 2 | 48 | ND |
| 77 | 1.82 (A) | 6 | 3 | 39 | 304 |
| 78 | 1.87 (A) | 5 | 3 | 61 | 355 |
| 79 | 1.63 (A) | 5 | 2 | 27 | 284 |
| 80 | 1.67 (A) | 2 | 2 | 40 | 375 |
| 81 | 2.55 (**) | 0.3 | 4 | 4 | 24 |
| 82 | 2.75 (A) | 0.05 | 6 | 1 | 2 |
| 83 | 1.68 (A) | 54 | 3 | 6 | ND |
| 84 | 1.63 (A) | 13 | 4 | 9 | ND |
| 85 | 1.86 (A) | 1 | 1 | 3 | 84 |
| 86 | 1.98 (A) | 1 | 3 | 2 | 27 |
| 87 | 2.2 (A*) | 0.1 | 14 | 1 | 5 |
| 88 | 2.11 (A) | 0.06 | 3 | 1 | ND |
| 89 | 1.78 (A) | 18 | 3 | 75 | ND |
| 90 | 1.57 (A) | 1 | 2 | 6 | 65 |
| 91 | 1.76 (A) | 1 | 5 | 2 | 5 |
| 92 | 1.89 (A) | 1 | 5 | 2 | 3 |

1. A peptide comprising at least 24 contiguous amino acids linked to a N-capping group wherein said N-capping group is selected from the group succinyl, acetyl, butanoyl, pentanoyl, hexanoyl or isovaleryl and wherein the first of said 24 amino acids is either directly linked to the N-capping group or is indirectly linked to said N-capping group via an additional amino acid selected from the group E, A or a and wherein
the first amino acid is C, Hcy, C(Bzl) or N,
the second amino acid is Y,
the third amino acid is a lipophilic amino acid,
the fourth amino acid represents A or R,
the fifth amino acid is C, Hcy or L,
the sixth amino acid is I,
the seventh amino acid is an acidic amino acid,
the eighth amino acid represents alanine or an acidic amino acid,
the ninth amino acid is L,
the tenth amino acid is a lipophilic amino acid,
the eleventh amino acid is a basic amino acid,
the twelfth amino acid is alanine or a basic amino acid,
the thirteenth amino acid is a lipophilic amino acid,
the fourteenth amino acid is Q or R,
the fifteenth amino acid is E,
the sixteenth amino acid is Q,
the seventeenth amino acid is Q,
the eighteenth amino acid is E,
the nineteenth amino acid is K,
the twentieth amino acid is N,
the twenty-first amino acid is E,
the twenty-second amino acid is A,
the twenty-third amino acid is a lipophilic amino acid and
the twenty-fourth amino acid is L;
optionally said twenty-fourth amino acid is linked to an amino acid selected from the group R, r, L, Tba or K (palmitoyl)

2. A peptide according to claim 1 wherein
the third amino acid is selected from the group A, L, I, F, V, W, Tba, Nva, Abu or Cha,
the fourth amino acid is R or A,
the seventh amino acid is selected from E or D,
the eighth amino acid, when an acidic amino acid, represents E,
the tenth amino acid is selected from I or Tba,
the eleventh amino acid, when a basic amino acid, is R or K,
the twelfth amino acid, when a basic amino acid, is R or K,
the thirteenth amino acid is selected from A, Nva or Abu, and
the twenty-third amino acid is A or L.

3. A peptide according to claim 1 wherein the first amino acid and fifth amino acid independently from one another are either C or Hcy and wherein said first and said fifth amino acid are connected via B1, B2, B3, B21 or B22.

4. A peptide according to claim 1 wherein the amino acid R, as linked to the twenty-fourth amino acid, is indirectly attached to cholesterol or palmitoyl or their derivatives thereof.

5. A peptide according to claim 4 having a linker between said amino acid R and said cholesterol or palmitoyl or their derivatives thereof, wherein said linker comprises two or more amino acids, preferably wherein the linker is
-Gly-Ser-Gly-Cys- (-GSGC-) or -Gly-Ser-Gly-Lys (-GSGK-).

6. The peptide according to claim 1 wherein amino acid sequences are in a dimer or trimer configuration

7. A peptide according to claim 1 having the amino acid sequence selected from the group: Pentanoyl E C Y L A C I-E-A-L-I-R-A-A-Q-E-Q-Q-E-K-N-E-A-A-L-R-NH₂, Pentanoyl-E-C-Y-L-A-C-I-E-E-L-I-R-K-A-Q-E-Q-Q-E-K-N-E-A-A-L-R-NH₂ or Suc-ECYLRCIEELIRKAQEQQEKNEAALR-NH₂ wherein the cysteine (C) moieties in the peptide are connected via B1.

8. A peptide according to claim 1 having the amino acid sequence:
Isovaleryl-E-C(Bzl)Y-L-A-L-I-E-E-L-I-R-K-A-Q-E-
Q-Q-E-K-N-E-A-A-L-R-NH₂.

9. A peptide according to any of the claim 1 having the amino acid sequence selected from Suc-ECYLRCIEELIRKAQEQQEKNEAALRGSGC (cholesteryl-oxycarbonylmethyl)-NH₂ and Ac-ACYAACIEALIRAAQEQQEKNEAALRGSGC (cholesteryloxycarbonylmethyl)-NH₂ wherein the cysteine (C) moieties at position 1 and position 5 in the peptide are connected via B1.

10. A peptide according to any of the claim 1 having the amino acid sequence selected from
Suc-E-C-Y-L-R-C-I-E-E-L-I-R-K-A-Q-E-Q-Q-
E-K-N-E-A-A-L-R
GSGK(palmitoyl)-NH₂ wherein the cysteine (C) moieties at position 1 and position 5 in the peptide are connected via B1;
Suc-E-N-Y-L-R-L-I-E-E-L-I-R-K-A-Q-E-Q-Q-
E-K-N-E-A-A-L-R-
GSGC(cholesteryl-oxycarbonylmethyl)-NH₂
and
Suc-E-N-Y-L-R-L-I-E-E-L-I-R-K-A-Q-E-Q-Q-
E-K-N-E-A-A-L-R-
GSGK(palmitoyl)-NH₂

11. Use of the peptide of any of the claim 1 for the inhibition of the HIV fusion with, or HIV entry in, a host cell.

12. Use of the peptide of any of the claim 1 for the manufacture of a medicament to prevent or to treat HIV fusion with, or HIV entry in, a host cell or HIV infection in humans.

13. Pharmaceutical composition comprising the peptide having the amino acid sequence in accordance with any of the claim 1 together with a pharmaceutically acceptable carrier.
